(19) United States
(12) Patent Application Publication
(10) Pub. No.: US 2014/0031808 A1
(43) Pub. Date: Jan. 30, 2014

(54) MEDICAL DEVICE TRACKING AND ENERGY FEEDBACK

(71) Applicant: Boston Scientific Scimed, Inc., Maple Grove, MN (US)

(72) Inventors: Huy D. Phan, San Jose, CA (US); William Wizeman, Mountain View, CA (US); Gary Kaplan, Mountain View, CA (US); Noah D. Webster, San Francisco, CA (US)

(73) Assignee: Boston Scientific Scimed, Inc., Maple Grove, MN (US)

(21) Appl. No.: 13/895,082

(22) Filed: May 15, 2013

Related U.S. Application Data
(60) Provisional application No. 61/675,259, filed on Jul. 24, 2012.

(51) Int. Cl. A61B 19/00; A61B 18/18

(52) U.S. Cl. CPC A61B 19/5212; A61B 18/18; A61B 19/20; A61B 19/2203
USPC 606/33

(57) ABSTRACT

Devices and methods of treating tissue defining a passageway in a body are disclosed. The devices may include an energy delivery system including an energy delivery device having an elongate member that includes an energy emitting portion at the distal portion of the elongate member. The energy emitting portion may be configured to transition between a first collapsed configuration and a second, expanded configuration. In addition, the methods may include positioning the energy delivery device in the passageway, moving the energy delivery device along the passageway in a substantially continuous manner.

### CROSS-REFERENCE TO RELATED APPLICATIONS

This application claims the benefit of priority from U.S. Provisional Application No. 61/675,259, filed on Jul. 24, 2012, the entirety of which is incorporated by reference herein.

### TECHNICAL FIELD

Embodiments of the present disclosure relate generally to devices and methods for treating tissue in a cavity or passageway of a body. More particularly, embodiments of the present disclosure relate to devices and methods for tracking a catheter and treating tissue based on that tracking in an airway of a lung, among other things.

### BACKGROUND

The anatomy of a lung includes multiple airways. As a result of certain genetic and/or environmental conditions, an airway may become fully or partially obstructed, resulting in an airway disease such as emphysema, bronchitis, chronic obstructive pulmonary disease (COPD), and asthma. Certain obstructive airway diseases, including, but not limited to, COPD and asthma, are reversible. Treatments have accordingly been designed in order to reverse the obstruction of airways caused by these diseases.

One treatment option includes management of the obstructive airway diseases via pharmaceuticals. For example, in a patient with asthma, inflammation and swelling of the airways may be reversed through the use of short-acting bronchodilators, long-acting bronchodilators, and/or anti-inflammatories. Pharmaceuticals, however, are not always a desirable treatment option because in many cases they do not produce permanent results or patients are resistant or non-compliant when it comes to drug therapies.

Accordingly, more durable and effective treatment options have been developed in the form of energy delivery systems, which include energy delivery devices for reversing obstruction of airways. Such devices may be designed to contact an airway of a lung to deliver energy at a desired intensity for a period of time that allows for the airway tissue (e.g., smooth muscle, nerve tissue, etc.) to be altered and/or ablated. Often, however, treatment of an airway is required at multiple locations along a length of the airway, and these locations may or may not be known prior to insertion of an energy delivery device into the airway. There is accordingly a need for an energy delivery system that enables mapping, real-time tracking, and/or locating of an energy delivery device in order to treat multiple tissue locations within an airway of a body based on that tracking.

### SUMMARY OF THE DISCLOSURE

In accordance with the present disclosure, methods and systems for treating tissue defining a passageway in a body are disclosed. The method may include positioning an energy delivery device in the passageway. The energy delivery device may include an elongate member having an energy emitting portion at a distal portion of the elongate member. In addition, the method may include contacting the tissue defining the passageway with the energy emitting portion, moving the energy delivery device along the passageway, real-time tracking a location and movement of the energy delivery device in the passageway, and delivering energy to the tissue according to the real-time tracked location and movement of the energy delivery device as it is moved along the passageway, such that at least one energy delivery parameter is automatically adjusted as the energy delivery device is moved along the passageway based on the real-time tracking.

The method may further include the following steps either alone or in combination: maintaining contact between the energy emitting portion and the tissue while the energy delivery device is moved along the passageway, and the real-time tracking may include generating an image of the passageway and imposing a real-time location of the energy delivery device on the image; the real-time tracking may include tracking the energy delivery device with a locating system and at least one locating implement, such that the at least one locating implement may be attached to the energy delivery device; the at least one energy delivery parameter may include at least one of power, temperature, impedance, and time; the energy emitting portion may include a plurality of legs forming a basket-type shape, with at least one energy delivery parameter being varied along a length of at least one of the legs; the at least one energy delivery parameter may also be automatically adjusted in response to sensed characteristics of the tissue and/or passageway; transitioning the energy emitting portion from a first, collapsed configuration to a second, expanded configuration and maintaining the energy emitting portion in the second, expanded configuration as the energy delivery device is moved along the passageway; moving the energy delivery device (manually or automatically via motor, robot, or the like) along the passageway in a substantially continuous manner (e.g., without being stationary at any given treatment site).

In addition, an energy delivery system for treating tissue defining a passageway in a body is disclosed. The energy delivery system may include an energy delivery device including an elongate member and an energy emitting portion at a distal portion of the elongate member, at least one energy generator connected to the energy delivery device, and a locating system configured to track the energy delivery device in real-time when the energy delivery device is within the passageway. The energy generator may be configured to deliver energy via the energy emitting portion to the passageway according to the real-time tracking of the energy delivery device, and the energy delivery system may be configured to automatically adjust at least one energy delivery parameter as the energy delivery device is moved along the passageway.

The energy delivery system may further include the following features either alone or in combination: maintain contact between the energy delivery device and tissue while the energy delivery device is moved along the passageway; the locating system may be configured to track the energy delivery device in real-time via communication with a locating implement located on the energy delivery device; the locating implement may be located on one of the elongate member, the energy emitting portion, or a distal tip of the energy delivery device; the energy emitting portion may include a plurality of legs forming a basket-type shape; the energy delivery system may be configured to vary the at least one energy delivery parameter along a length of at least one of the plurality of legs; the at least one energy parameter may include at least one of temperature, impedance, time, and power; there may be a rendering system configured to generate an image of coordinates of the passageway; the energy delivery system may be configured to also automatically adjust the at least one energy delivery parameter in response to measured characteristics of the tissue and/or passageway; the energy emitting portion may be self-expanding; the energy emitting portion may be configured to transition from a first, collapsed configuration to a second, expanded configuration, and the energy emitting portion may be configured to be maintained in the second, expanded configuration as the energy delivery device is moved along the passageway.

Additional objects and advantages of the disclosure will be set forth in part in the description which follows, and in part will be obvious from the description, or may be learned by practice of the disclosure. The objects and advantages of the disclosure will be realized and attained by means of the elements and combinations particularly pointed out in the appended claims.

The accompanying drawings, which are incorporated in and constitute a part of this specification, illustrate several embodiments of the present disclosure and together with the description, serve to explain the principles of the invention.

### BRIEF DESCRIPTION OF THE DRAWINGS

FIG. 1 is a schematic view of a system for delivering energy to tissue within a cavity or passageway of a body.

FIG. 2 is an enlarged view of a distal portion of an energy delivery device, according to a first embodiment of the present disclosure.

FIG. 3 is a schematic view of the system of FIG. 1 with an energy delivery device inserted into a cavity or passageway of a body.

FIG. 4 is a schematic view of a virtual map showing the virtual position of an energy delivery device within a cavity or passageway of a body.

### DESCRIPTION OF THE EMBODIMENTS

Reference will now be made in detail to exemplary embodiments of the present disclosure, examples of which are illustrated in the accompanying drawings. Wherever possible, the same reference numbers will be used throughout the drawings to refer to the same or like parts.

Embodiments of the present disclosure relate to devices and methods for applying energy to tissue within a wall or cavity of a body. More particularly, embodiments of the present disclosure relate to devices and methods for applying energy to tissue in the airway of a lung in order to treat reversible obstructive airway diseases including, but not limited to, COPD and asthma. It should be emphasized, however, that embodiments of the present disclosure may also be utilized in any procedure where heating of tissue is required, such as, for example cardiac ablation procedures.

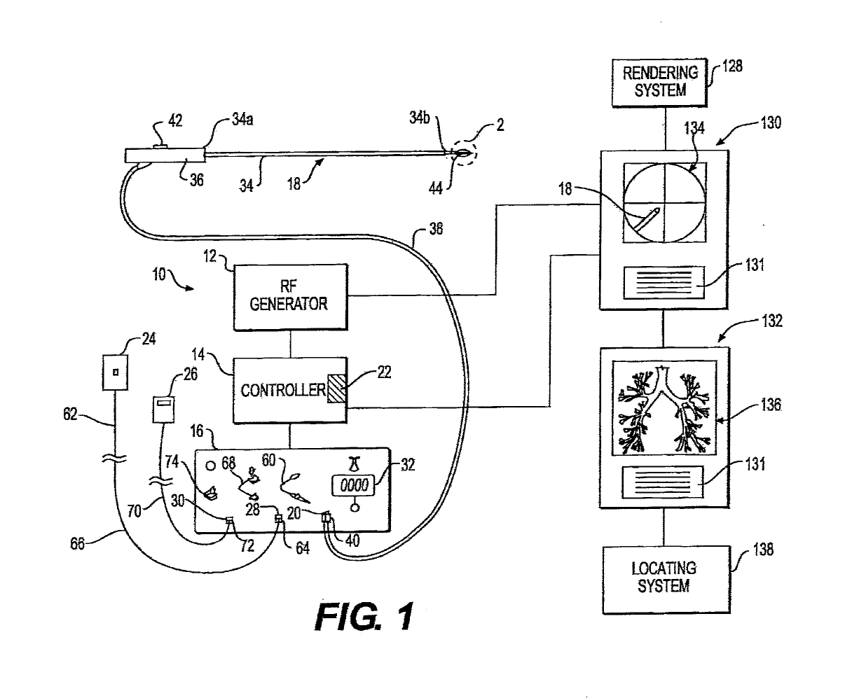

FIG. 1 illustrates a system 10 for delivering energy, in accordance with a first embodiment of the present disclosure. The system 10 may include an energy generator 12, a controller 14, a user interface surface 16, and an energy delivery device 18. Energy generator 12 may be any suitable device configured to produce energy for heating and/or maintaining tissue in a desired temperature range. In one embodiment, for example, energy generator 12 may be an RF energy generator. The RF energy generator may be configured to emit energy at specific frequencies, powers, and/or temperatures and for specific amounts of time in order to reverse obstruction in an airway of a lung.

In certain obstructive airway diseases, obstruction of an airway may occur as a result of narrowing due to airway smooth muscle contraction. Accordingly, in one embodiment, energy generator 12 may be configured to emit energy that reduces the ability of the smooth muscle to contract and/or increases the diameter of the airway by debulking, denaturing, and/or eliminating the smooth muscle or nerve tissue. That is, energy generator 12 may be configured emit energy capable of killing smooth muscle cells or nerve tissue, preventing smooth muscle cells or nerve tissue from replicating, and/or eliminating smooth muscle or nerve tissue by damaging and/or destroying the smooth muscle or nerve tissue.

More particularly, energy generator 12 may be configured to generate energy with a wattage output sufficient to maintain a target tissue temperature in a range of about 60 degrees Celsius to about 80 degrees Celsius. In one embodiment, for example, energy generator may be configured to generate RF energy at a frequency of about 400 kHz to about 500 kHz and for treatment cycle durations of about 5 seconds to about 15 seconds per treatment cycle. Alternatively, the duration of each treatment cycle may be set to allow for delivery of energy to target tissue in a range of about 125 Joules of RF energy to about 150 Joules of energy. In one embodiment, for example, when a monopolar electrode is used, the duration of treatment may be about 10 seconds, and the target tissue temperature may be about 65 degrees Celsius. In another embodiment, when a bipolar electrode is used, the duration of treatment may be about 2 to 3 seconds, with the target tissue temperature being approximately 65 degrees Celsius.

Energy generator 12 may further include, or be coupled to, an energy operating mechanism 26. Energy operating mechanism 26 may be any suitable automatic and/or user operated device in operative communication with energy generator 12 via a wired or wireless connection, such that energy operating mechanism 26 may be configured to enable activation of energy generator 12. Energy operating mechanism 26 may therefore include, but is not limited to, a switch, a push-button, or a computer. The embodiment of FIG. 1, for example, illustrates that energy operating mechanism 26 may be a footswitch. The footswitch may be coupled to a conductive cable 70 that is, in turn, coupled to a proximal coupler 72 which is configured to be electrically coupled to an interface coupler 30 disposed on user interface surface 16.

Energy generator 12 may be coupled to controller 14. Controller 14 may include a processor 22 configured to receive information feedback signals, process the information feedback signals according to various algorithms, produce signals for controlling the energy generator 12, and produce signals directed to visual and/or audio indicators. For example, processor 22 may include one or more integrated circuits, microchips, microcontrollers, and microprocessors, which may be all or part of a central processing unit (CPU), a digital signal processor (DSP), an analogy processor, a field programmable gate array (FPGA), or any other circuit known to those skilled in the art that may be suitable for executing instructions or performing logic operations. That is, processor 22 may include any electric circuit that may be configured to perform a logic operation on at least one input variable. In one embodiment, for example, processor 22 may be configured to use a control algorithm to process a temperature feedback signal and generate control signals for energy generator 12. Alternative or additional control algorithms and system components that may be used in conjunction with processor 22 may be found in U.S. Pat. No. 7,104,987, titled CONTROL SYSTEM AND PROCESS FOR APPLICATION OF ENERGY TO AIRWAY WALLS AND OTHER MEDIUMS, issued Sep. 12, 2006, and in U.S. Patent Application Publication No. 2009/0030477 titled SYSTEM AND METHOD FOR CONTROLLING POWER BASED ON IMPEDANCE DETECTION, SUCH AS CONTROLLING POWER TO TISSUE TREATMENT DEVICES, published on Jan. 29, 2009, each of which is incorporated by reference herein in its entirety.

Controller 14 may additionally be coupled to and in communication with user interface 16. The embodiment of FIG. 1 illustrates that controller 14 may be electrically coupled to user interface 16 via a wire connection. In alternative embodiments, however, controller 14 may be in wireless communication with user interface 16. User interface 16 may be any suitable device capable of providing information to an operator of the energy delivery system. Accordingly, user interface 16 may be configured to operatively couple to each of the components of energy delivery system 10, receive information signals from the components, and output at least one visual or audio signal to a device operator in response to the information received. The surface of user interface 16 may therefore include, but is not limited to, at least one switch 74, a digital display 32, visual indicators, audio tone indicators, and/or graphical representations of components of the energy delivery system 60, 68. Embodiments of user interface 16 may be found in U.S. Patent Application Publication No. 2006/0247746 A1, titled CONTROL METHODS AND DEVICES FOR ENERGY DELIVERY, published Nov. 2, 2006, which is incorporated by reference herein in its entirety.

FIG. 1 illustrates that user interface 16 may be coupled to energy delivery catheter 18. The coupling may be any suitable medium enabling distribution of energy from energy generator 12 to energy deliver device 18, such as, for example, a wire or a cable 38. Energy delivery device 18 may include an elongate member 34 having a proximal end 34a and a distal end 34b. Elongate member 34 may be any suitable longitudinal device configured to be inserted into a passageway of a body. The passageway may be any opening, lumen, or cavity within a body that is capable of receiving energy delivery device 18. Accordingly, elongate member 34 may further include any suitable stiff or flexible material configured to enable movement of energy delivery device 18 through a passageway in a body. In one embodiment, for example, elongate member 34 may be sufficiently flexible to enable elongate member to conform to the passageway through which it is inserted.

Elongate member 34 may be any suitable size, shape, and or configuration such that elongate member 34 may be configured to pass through a lumen of a bronchoscope. Elongate member may be solid or hollow. In one embodiment, for example, elongate member 34 may include one or more lumens or internal channels (not shown) for the passage of an actuation/pull wire 50 (as shown) and/or a variety of surgical equipment, including, but not limited to, locating system, imaging devices, tools for irrigation, insufflation, vacuum suctioning, biopsies, and drug delivery. Elongate member 34 may further include an atraumatic exterior surface having a rounded shape and/or coating. The coating may be any coating known to those skilled in the art enabling ease of movement of energy delivery device 18 through a passageway within a body. The coating may therefore include, but is not limited to, a lubricious coating and/or an anesthetic.

FIG. 1 further illustrates that an energy emitting portion 44 may be attached to distal end 34b of elongate member 34. Energy emitting portion 44 may be permanently or removably attached to distal end 34b of elongate member. In one embodiment, for example, energy emitting portion 44 may be permanently or removably attached to elongate member 34 via a flexible junction enabling movement of energy emitting portion 44 relative to distal end 34b of elongate member 34. Embodiments of a junction may be found, for example, in U.S. Patent Application Publication No. 2006/0247618 A1 titled MEDICAL DEVICE WITH PROCEDURE IMPROVEMENT FEATURES, published Nov. 2, 2006, which is incorporated by reference herein in its entirety.

Energy emitting portion 44 may be any suitable device configured to emit energy from energy generator 12. In addition, energy emitting portion 44 may include a contact region 102 that may be configured to contact tissue 104 within a passageway 100 of a body. The contact region 102 may include at least a portion that is configured to emit energy from energy generator 12. Energy emitting portion 44 may further be a resilient member configured to substantially maintain a suitable size, shape, and configuration that corresponds to a size of a passageway in which energy delivery device 18 is inserted.

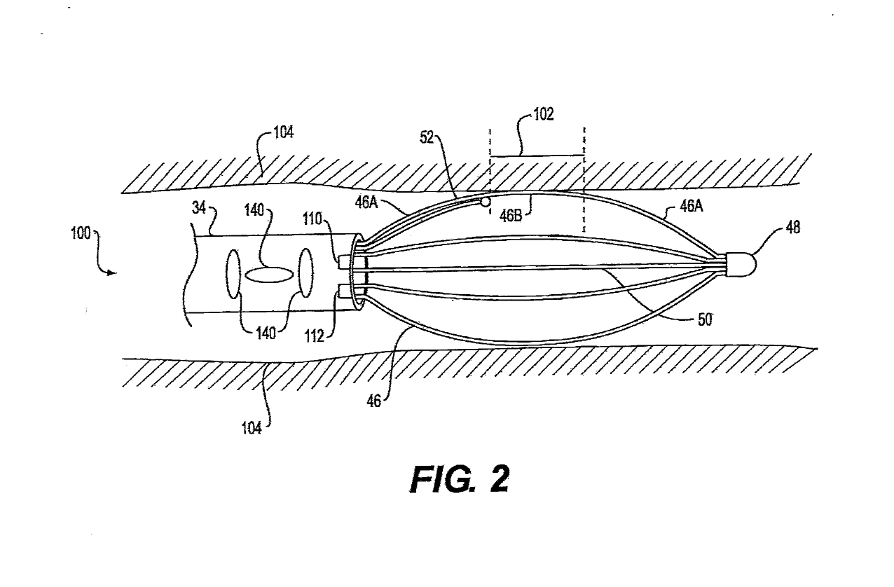

In one embodiment, for example, energy emitting portion 44 may be an expandable member. The expandable member may include a first, collapsed configuration (not shown) and a second, expanded configuration (FIG. 2). The expandable member may include any size, shape, and/or configuration, such that in the second, expanded configuration, the contact region may be configured to contact tissue in a passageway of a body. The expandable member of energy emitting portion 44 may be any suitable expandable member known to those of skill in the art including, but not limited to, a balloon or cage. In one embodiment, as illustrated in FIGS. 1 and 2, energy emitting portion 44 may include an expandable basket having a plurality of legs 46 configured to converge at an atraumatic distal tip 48 of energy delivery device 18.

FIG. 2 illustrates that energy emitting portion 44 may be configured to expand to form a basket having a substantially symmetrical shape in a cavity and/or passageway 100 of a body having a substantially constant diameter along the length of energy emitting portion 44. Energy emitting portion 44, however, may be configured to expand to a non-symmetrical shape such that energy emitting portion 44 may be configured to be selectively expanded along its length in order to conform to a passageway in a body having a variable diameter along the length of energy emitting portion 44.

Energy emitting portion 44 may be configured to be biased towards the first, collapsed configuration. For example, in order to transition between the first, collapsed configuration and the second, expanded configuration, a force must be exerted on either a proximal or distal end of energy emitting portion 44 in order to cause each of the plurality of legs 46 to bow radially outward. In an alternative embodiment, however, energy emitting portion 44 may be self-expandable, such that energy emitting portion 44 is normally biased to the second, expandable configuration. Accordingly, energy delivery device 18 may further include a retractable sheath (not shown) or other suitable retractable member configured to maintain energy emitting portion 44 in the first, collapsed configuration. Upon retraction of the sheath in a distal direction, energy emitting portion 44 may be configured to self-expand and transition from the first, collapsed configuration to the second, expandable configuration.

As previously discussed, FIGS. 1 and 2 illustrate that upon expansion of energy emitting portion 44, each of the plurality of legs 46 may be configured to bow radially outward from a longitudinal axis of energy delivery device 18. A central portion 46B of each leg 46 may be configured to be the portion of the leg 46 that is the greatest distance from the longitudinal axis when the energy emitting portion 44 is in its second, expanded configuration. As illustrated in FIG. 2, in some embodiments, the central tissue-contacting portion 46B of at least one leg 46 may form a rounded or curved configuration upon expansion of energy emitting portion 44. Alternatively, the central portion 46B of at least one leg 46 may be configured to maintain a substantially straight or linear configuration upon expansion of energy emitting portion 44, such that energy delivery device 18 may be configured to provide more uniform and controlled energy delivery to tissue in a passageway within a body.

Central portion 46B of each leg 46 may accordingly be any suitable size (e.g., length), shape, and/or configuration. In some embodiments, for example, each leg 46 may have a substantially similar central portion 46B. Alternatively, in other embodiments, the central portion 46B of each leg 46 may vary. The size, shape, and/or configuration of the central portion 46B may be determined based on multiple factors including, but not limited to, the location of treatment, desired treatment energy level, duration of treatment, and size of body lumen.

Legs 46 of energy emitting portion 44 may further include an atraumatic exterior surface, so that energy emitting portion 44 may not cause unwanted damage of tissue within a passageway of a body as energy delivery device 18 is moved through the passageway. In addition, at least a portion of an exterior surface of each leg 46 may include a lubricious and/or anesthetic insulation. The insulation may be any suitable material known to those skilled in the art enabling ease of movement of energy delivery device 18 through a passageway within a body.

Energy emitting portion 44 may further include at least one electrode. The at least one electrode may be any suitable configured to emit energy, including such electrodes known to those skilled in the art. The at least one electrode may be located along the length of at least one of the plurality of legs 46 or comprise at least one of the plurality of legs 46 and may include at least a portion of the contact region 102 of energy emitting portion 44. Accordingly, the at least one electrode may include, but is not limited to, at least one band electrode, ball electrode or a dot electrode or a plurality thereof.

In one embodiment, for example, at least one leg 46 may include a plurality of electrodes along its length with each electrode being attached to the same energy generator 12. In an alternative embodiment, energy delivery system may include at least two energy generators 12, such that at least two of the plurality of electrodes on the at least one leg 46 are connected to different energy generators 12. Accordingly, energy delivery variables including, but not limited to, time, power, and temperature, may be varied along the length of the at least one leg 46 during treatment of a passageway in a body. Alternatively, the embodiment of FIG. 1 illustrates that at least one leg 46 of the energy emitting portion is made up of a single, elongate electrode 46. In one embodiment, for example, the elongate electrode 46A may have an electrical insulator material. In addition, at least a portion of the electrode 46B may be exposed, forming an active region for delivering energy to tissue.

The at least one electrode may be monopolar or bipolar. The embodiment of FIG. 1 illustrates an energy emitting portion 44 including monopolar electrodes. Accordingly, the embodiment of FIG. 1 further includes a return electrode component 62 configured to complete an electrical energy emission or body circuit between energy generator 12 and a body (not shown). Return electrode component 62 may include a conductive pad 24, a proximal coupler 64, and a conductive cable 66 extending between and in electrical communication with conductive pad 24 and proximal coupler 64.

Conductive pad 24 may include a conductive adhesive surface configured to removably stick to a patient's skin. In addition, conductive pad 24 may include a surface area having a sufficient size in order to alleviate burning or other injury to the patient's skin that may occur in the vicinity of the conductive pad 24 during energy emission. Moreover, proximal coupler 64 may be configured to couple to an interface coupler 28 on user interface surface 16. As illustrated in FIG. 1, interface coupler 28 may be disposed adjacent a graphical representation 68 of the electrode component 62 on the user interface surface 16, such that user interface surface 16 may be configured to provide at least a visual indicator in relation to return electrode component 62.

Energy delivery device 18 may further include a handle 36. Handle 36 may be any suitable handle known to those skilled in the art that is configured to enable a device operator to control movement of energy delivery device 18 through a body. In addition, in some embodiments, handle 36 may further be configured to control expansion of energy emitting portion 44.

A rod, cable, or wire may be located within handle 36 and may extend through elongate member 34, connecting to energy emitting portion 44. Actuation of handle 36 may cause the rod, cable, or wire to move and exert a force on energy emitting portion 44, which may thereby cause expansion or contraction of energy emitting portion 44. In one embodiment, for example, a push rod may extend from handle 36 and be connected to a proximal end of energy emitting portion 44. Actuation of handle 36 may allow for distal movement of the push rod, which may exert a distal force on the proximal end of energy emitting portion 44. The force on the proximal end of energy emitting portion 44 may cause each of the plurality of legs 46 to bow radially outward, thereby expanding energy emitting portion 44. Alternatively, as illustrated in FIG. 2, a pull wire 50 may extend from handle 36 and connect to distal tip 48. Actuation of handle 36 may exert a proximal force of pull wire 50, thereby causing the plurality of legs 46 to bow outward and expand energy emitting portion 44.

Handle 36 may accordingly include an actuator mechanism, including, but not limited to, a squeeze handle, a foot pedal, a switch, a push button, a thumb wheel, or any other suitable actuation device known to those skilled in the art. The embodiment of FIG. 1, for example, illustrates that the actuator mechanism may be a sliding actuator 42. Sliding actuator 42 may be connected to pull wire 50 and may be any suitable device known to those skilled in the art configured to move along handle 36 in both the proximal and distal directions. Sliding actuator 42 may additionally include at least one of a stop and/or a locking mechanism. In one embodiment, for example, sliding actuator 42 may be ratcheted. Alternatively, sliding actuator 42 may be configured to slide freely in both the proximal and distal directions until being acted on by one of the stop and/or locking mechanism. A further embodiment of handle 36 may be found in U.S. Patent Application Publication No. 2009/0018538 titled SYSTEMS AND METHODS FOR DELIVERING ENERGY TO PASSAGEWAYS IN A PATIENT, published on Jan. 15, 2009, which is incorporated by reference herein in its entirety.

Energy delivery device 18 may further include at least one sensor (not shown) configured to be in wired or wireless communication with the display and/or indicators on user interface surface 16. In one embodiment, for example, the at least one sensor may include a wire extending through elongate member 34 and handle 36, and being operatively connected to cable 38.

The at least one sensor may be configured to sense tissue temperature and/or impedance level. In one embodiment, as illustrated in FIG. 2, energy emitting portion 44 may include at least one temperature sensor 52 in the form of a thermocouple. Embodiments of the thermocouple may be found in U.S. Patent Application Publication No. 2007/0100390 A1, titled MODIFICATION OF AIRWAYS BY APPLICATION OF ENERGY, published May 3, 2007, which is incorporated by reference herein in its entirety.

In addition, the at least one sensor may be configured to sense functionality of the energy delivery device. For example, the at least one sensor may be configured to sense the placement of the energy delivery device within a body, whether components are properly connected, whether components are properly functioning, and/or whether components have been placed in a desired configuration. In one embodiment, for example, energy emitting portion 44 may include a pressure sensor or strain gauge for sensing the amount of force energy emitting portion 44 exerts on tissue in a passageway in a body. The pressure sensor may be configured to signal energy emitting portion 44 has been expanded to a desired configuration such that energy emitting portion 44 may be prevented from exerting a damaging force on surrounding tissue or the device 44 (e.g., electrode inversion) or not enough force indicating that improved tissue electrode contact is needed for improved performance. Accordingly, the at least one sensor may be placed on any suitable portion of the energy delivery device including, but not limited to, on energy emitting portion 44, elongate member 34, and/or distal tip 48.

In addition, energy delivery device 18 may include at least one imaging device 110 located on one of the energy emitting portion 44, elongate member 34, and/or distal tip 48. The imaging device 110 may include a camera or any other suitable imaging device known to those skilled in the art and configured to transmit images to an external display. The energy delivery device 18 may additionally include at least one illumination source 112. The illumination source 112 may be integrated with the imaging device 110, as illustrated in FIG. 2, or a separate structure attached to one of the energy emitting portion 44, elongate member 34, and/or distal tip 48. The illumination source may provide light at a wavelength for visually aiding the imaging device. Alternatively, or in addition, the illumination source may provide light at a wavelength that allows a device operator to differentiate tissue that has been treated by the energy delivery device from tissue that that not been treated.

Additional embodiments of the imaging or mapping device may be found in U.S. Patent Application Publication Nos. 2006/0247617 A1, titled ENERGY DELIVERY DEVICES AND METHODS, published Nov. 2, 2006; and 2010/0268222 A1, titled DEVICES AND METHODS FOR TRACKING AN ENERGY DEVICE WHICH TREATS ASTHMA, published Oct. 21, 2010, each of which are incorporated by reference herein in its entirety.

The energy delivery system 10 of FIG. 1 further includes an energy delivery device tracking system which may be configured for real-time tracking, mapping, and/or locating of energy delivery device 18. The energy delivery device tracking system may include, but is not limited to, visual displays 130, 132, a locating system 138, locating implements 140, a rendering system 128, and at least one sensor.

Rendering system 128 may be configured to generate an image of a desired location within a body. In addition, or alternatively, rendering system 128 may be configured to produce a virtual map or a series of coordinates relative to a fixed reference point somewhere in the body. In the embodiment of FIG. 1, for example, rendering system 128 may be configured to generate both an image of a system of cavities and/or passageways within a body 134 as well as a virtual map 136. Accordingly, rendering system may be any imaging system known to those skilled in the art, including, but not limited to, computer aided tomography (CT), magnetic resonance imaging (MRI), positron emission tomography (PET), ultrasonic imaging, optical coherence tomography (OCT), electromagnetic or acoustic waves, or any other suitable imaging system known to those skilled in the art.

Rendering system 128 may configured to be located external to a body (e.g., a CT, MRI, PET, etc.). Alternatively, rendering system 128 may include a probe or other similar imaging component and may be configured to generate an image and/or coordinates as it travels through a body. Accordingly, rendering system 128 may be a device that is independent from energy delivery device 18. Alternatively, rendering system may be removably or permanently fixed to one of elongate member 34, energy emitting portion 44, or distal tip 48 of energy delivery device 18.

Rendering system 128 may further be in wired or wireless communication with at least one visual display 130, 132. Visual displays 130, 132 may include an image of the body, a map or series of coordinates, a treatment location, and/or a location of energy delivery device 18. In addition, each of the visual displays 130, 132 may be configured to produce a two-dimensional or three-dimensional image, map, etc. The embodiment of FIG. 1 illustrates that each visual display 130, 132 displays an image of either the body or a map/series of coordinates. Alternatively, visual displays 130, 132 may be combined such that a series of coordinates is displayed on the same screen as an image of the body. Accordingly, visual displays 130, 132 may be configured to enable a device operator to identify a treatment location, identify a location of energy delivery device 18, and/or direct energy delivery device 18 to a desired location within the body.

One or more of the visual displays 130, 132 may further be configured to display one or more treatment and information parameters. In one embodiment, for example, one or more treatment and information parameters may be displayed on the image 134 or map/series of coordinates 136. Alternatively, or in addition, as illustrated in FIG. 1, visual displays 130, 132 may be configured to display one or more treatment parameters on an information display screen 131. The one or more treatment and information parameters may include, but are not limited to, location of energy delivery device 18, time or duration of treatment, temperature (of the site, the device, or an adjacent site), energy, power, average power, functionality of energy delivery device 18, position of treatment, total energy applied, rate of change in temperature, rate of change in applied energy, impedance of the treatment location, size of the treatment location, combinations thereof, or any other suitable parameter or information known to those skilled in the art for aiding in energy delivery treatment.

In addition, at least one visual display 130, 132 may be configured to visually differentiate treated areas of the body from untreated areas of the body. For example, in one embodiment, virtual image 136 may be configured to differentiate treated areas from untreated areas and from areas that have been partially treated by automatically shading an area (e.g., with a color, design, etc.) of the image according to the stage of treatment received. Accordingly, the virtual image 136 may also be configured to provide visual information to allow a device operator to guide energy delivery device 18 to another treatment site.

Visual displays 130, 132 may be in wired or wireless communication with user interface 16. Accordingly, information received by user interface 16 may also be displayed on positioning information display 131. Visual displays 130, 132 may further be in wired or wireless communication with at least one of energy generator 12 and controller 14, such that visual displays 130, 132 may be configured to transmit the one or more treatment and information parameters to one or more components of energy delivery system 10, including, but not limited to, energy generator and/or controller 14. The transmittal of the one or more treatment and information parameters may enable instant, real-time energy control of energy emitting portion 44 of energy delivery device 18.

In addition, or alternatively, the transmittal of the one or more treatment and information parameters may enable controller 14 to generate and record a treatment history profile. Embodiments of the treatment history profile may be found in U.S. Pat. No. 7,594,925, titled CONTROL SYSTEMS FOR DELIVERING ENERGY, issued on Sep. 29, 2009, U.S. Pat. No. 7,708,768, titled DEVICES AND METHODS FOR TRACKING AN ENERGY DELIVERY DEVICE WHICH TREATS ASTHMA, issued on May 4, 2010, and U.S. Patent Application Publication No. 2010/0268222 A1, titled DEVICES AND METHODS FOR TRACKING AN ENERGY DEVICE WHICH TREATS ASTHMA, published Oct. 21, 2010, each of which is incorporated by reference herein in its entirety.

Generation of the treatment history profile as described herein may allow a device operator to potentially avoid overlapping treatment locations or over-treating a particular location. For example, in one embodiment, the treatment system may be configured to prevent providing therapy to a particular site if the image and/or map and associated parameter(s) indicate that energy delivery device 18 is in a location that was previously treated. In such cases, audio or visual signals may be shown on either visual display 130, 132 and/or on user interface 16, and controller 14 may receive a signal to temporarily or permanently turn off energy generator 12. Likewise, the treatment history profile also allows the device operator to avoid under-treatment.

In addition to being configured to generate a treatment history profile, energy delivery system 10 may be configured to generate an ideal procedure profile. The ideal procedure profile may include, but is not limited to, an ideal treatment time, ideal temperature, ideal energy, ideal rate of change in temperature, ideal rate of change in energy, ideal impedance of the treatment location, and any combination thereof. Accordingly, the system may allow for comparison of an ideal procedure to an actual treatment procedure during or after treatment.

The ideal procedure profile may be determined based on characteristics of the body identified in the virtual map 136 and/or image 134. Characteristics may include, but are not limited to, bifurcations, amount of smooth muscle tissue, depth of smooth muscle tissue, nerve tissue location, degree of smooth muscle contraction when smooth muscle is stimulated, diameter of passageway, branching points of a passageway, wall thickness of a passageway, proximity of the passageway relative to another portion of the body, periphery of a passageway, degree of fluids in a passageway, number of folds in a passageway, condition of an epithelial or endothelial layer in a passageway, fluid flow in a passageway, and presence of additional tissue, vessels, or cartilage in a passageway.

Returning to FIGS. 1 and 2, energy delivery device tracking system additionally includes a locating system 138 and at least one locating implement 140. Locating system 138 may be in wireless communication with the at least one locating implement 140, and may be configured to track and/or identify the location of at least one locating implement 140. Accordingly, locating system 138 may be any suitable locating system 138 known to those skilled in the art, including, but not limited to optical or electromagnetic locating systems. The at least one locating implement 140 may also be any suitable locating implement configured to communicate with locating system 138 (e.g., electromagnetic implement or optical computer mouse). In one embodiment, for example, the at least one locating implement 140 may be part of a sensor assembly that includes, but is not limited to, a light emitting source, a sensor, and a digital signal processor. Additional examples of such locating implements and locating systems are found in the references cited and incorporated by reference above.

The at least one locating implement 140 may be configured to be positioned on any suitable portion of energy delivery device 18. Accordingly, locating system 138 may be configured to track and/or identify the location of energy delivery device 18. The at least one locating implement 140 may be removably or permanently positioned or affixed on at least one of elongate member 34, energy emitting portion 44, and/or distal tip 48 to eliminate the need for a separate structure, and thereby reduce complexity and costs of system 10. As illustrated in FIG. 2, a plurality of locating implements 140 may be configured to be positioned on a distal portion of elongate member 34. Alternatively, or in addition, the at least one locating implement 140 may be configured to attach to a device external to energy delivery device 18, such as, for example, a bronchoscope, endoscope, and/or accompanying guidewire.

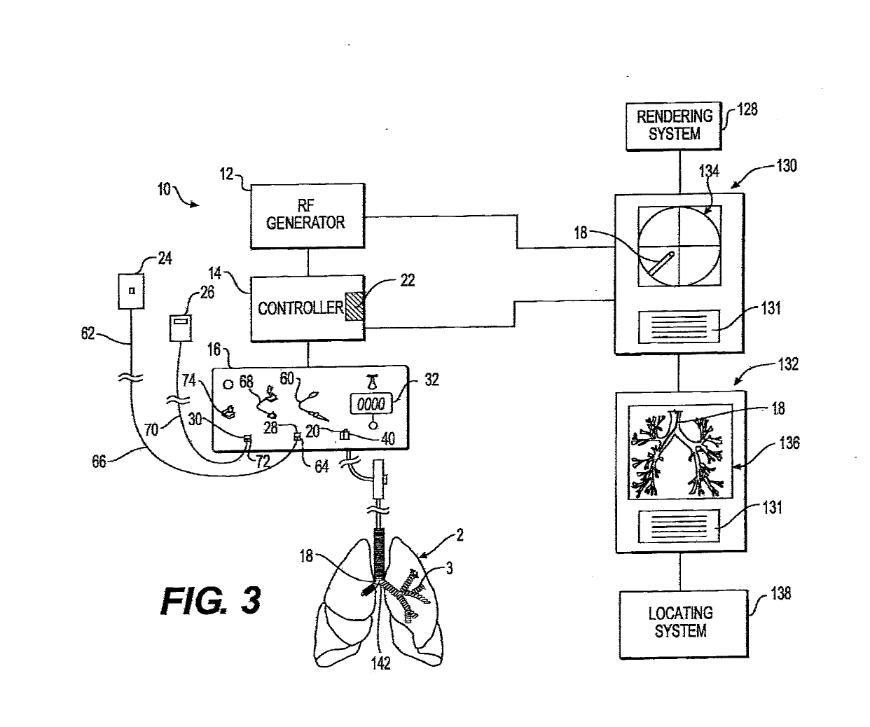

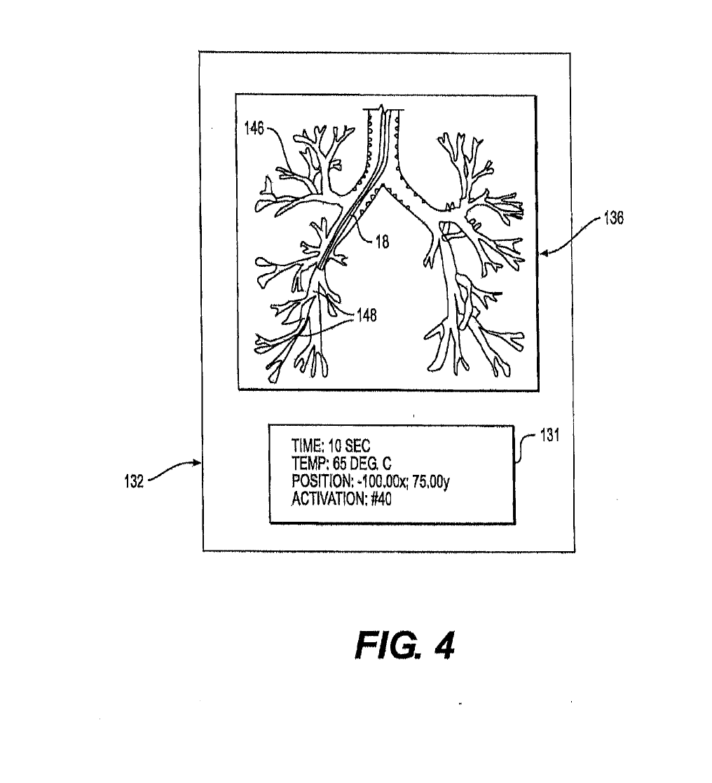

Moreover, locating system 138 may be configured to be in wired or wireless communication with at least one of the visual displays 130, 132, such that locating system 138 may be configured to provide real-time mapping and/or position information of the energy delivery device 18 within a body. FIG. 3 illustrates the energy delivery system 10 of FIG. 1 with energy delivery device 18 inserted in a system of airways 3 in a lung 2 of a body. In use, as energy delivery device 18 is advanced through the airways 3, locating system 138 may be configured to communicate with the at least one locating implement 140 and transmit the real-time location information of energy delivery device 18 within the airways 3, (for example, through bronchiole 146 branches 148), such that at least one of the visual displays 130, 132 can display a real-time virtual position of energy delivery device 18 on at least one of the real time image 134 and virtual map 136 generated by rendering system 128 (see FIGS. 3 and 4).

In one embodiment, once energy delivery device 18 is at a treatment site, the device operator may actuate handle 36 in order to transition energy emitting portion 44 from the first, collapsed configuration to the second, expanded configuration (FIG. 2). Alternatively, the device operator may transition energy emitting portion 44 from the first, collapsed configuration to the second, expanded configuration by retracting a sheath in the proximal direction. FIG. 2 illustrates that upon expansion, a portion 102 of at least one of the legs 46 may be in contact with tissue 104 of a passageway 100. Device operator may then turn on energy generator 12 (e.g., via foot pedal 26) in order to enable energy to be emitted from energy emitting portion 44. Upon completion of successful treatment at the treatment site, the device operator may turn off energy generator 12, release actuator 42 on handle 36 and/or move the sheath in the distal direction to return energy delivery device 18 to the first, collapsed configuration, and move energy delivery device 18 to another treatment site. This process may be repeated until all treatment sites along a passageway have been successfully treated. Upon completion of successful treatment of the passageway, a device operator may remove energy delivery device 18 from the body.

As previously discussed, energy delivery system 10 may be configured to map a passageway within a body and track energy delivery device 18 as energy delivery device 18 moves through the passageway. In addition, energy delivery system 10 may be configured to measure and record treatment parameters as energy delivery device 18 moves through the passageway, and compare the treatment parameters to an ideal procedure. Energy delivery system 10 may accordingly be configured to automatically adjust energy delivery based on the location of energy delivery device 18 as a result of a treatment plan determined by the ideal treatment history profile. In addition, or alternatively, energy delivery system 10 may be configured to automatically adjust at least one treatment parameter based on the location of energy delivery device 18 and/or based on the measured and recorded treatment parameters, as energy delivery device 18 is moved through the passageway. This automatic adjustment may allow for sufficient treatment along the passageway while mitigating the possibility of excessive or inadequate energy delivery to the passageway.

More specifically, energy delivery device 18 may be configured to treat multiple treatment sites along a passageway in a body without returning energy emitting portion to the first, collapsed configuration in between treatment sites. That is, energy delivery system 10 may be configured to treat multiple treatment sites along a passageway in a body as a device operator moves energy delivery device 18 along the passageway, while maintaining the energy delivery portion 44 in contact with a wall of the passageway. The device operator may also move energy delivery device 18 in a substantially continuous manner at a steady or unsteady rate. Alternatively, the device operator may move energy delivery device 18 in a discontinuous manner at a steady or unsteady rate.

Accordingly, in use, energy delivery system 10 may be configured to track energy delivery device 18 in real-time, and additionally energy delivery system 10 may be configured to automatically control the delivery of energy (time, power, etc.) from energy generator 12 based on the location of energy delivery device 18 as energy delivery device 18 is moved along a passageway in a body. For example, as previously discussed, at least one leg 46 of energy emitting portion 44 may be configured to have varying treatment parameters (e.g., time, temperature, power, impedance, etc.) along the length of at least one leg 46. Accordingly, energy delivery system 10 may be configured to automatically adjust the energy delivery treatment parameters along the length of the at least one leg 46 as energy delivery device 18 is moved through a passageway in a body, such as in a continuous, steady fashion. In addition, or alternatively, energy delivery system 10 may be configured to automatically adjust each leg 46 of the plurality of legs on a leg by leg basis. For example, as energy delivery device 18 is moved throughout a body, it may be desired to vary the amount of energy that each leg 46 emits as a result of the positioning of energy delivery device 18 within the body.

Moreover, in an embodiment where at least one leg 46 includes a single, elongate electrode, energy delivery system 10 may be configured to automatically adjust at least one energy delivery parameter of the entire electrode as energy delivery device 18 is moved through a passageway in a body. The automatic adjustment of at least one energy delivery parameter may be in response to continuously measured characteristics of the passageway, including, but not limited to, temperature, impedance, amount of energy delivered, length of time of energy delivery, and diameter of passageway.

In use, a device operator may continuously or discontinuously move energy delivery device 18 at a steady speed or a varying speed. Energy delivery system 10 may therefore include at least one sensor for monitoring the rate at which energy delivery device 18 is moved throughout a passageway. The at least one sensor may be in communication with the energy delivery device tracking system and/or the controller 14, such that energy delivery system 10 may be configured to emit audio and/or visual signals/warnings if energy delivery device 18 is being moved too quickly, causing unsuccessful treatment of a passageway, or if energy delivery device 18 is being moved too slow, causing overtreatment of a passageway.

The present disclosure provides for improved customized energy delivery to a plurality of tissue treatment sites. In particular, tailored treatment is may be enabled as a result of the electromagnetic or optical sensor on energy delivery device 18, which may be configured to continuously monitor movement and/or the location of energy delivery device 18 and correlate this information back to energy generator 12 so as to continuously control at least one energy parameter (power, time, temperature, impedance, etc.) applied to the passageway. For example, in one embodiment, if energy delivery portion 44 is moved proximally at a rate of 1 mm per second, such that the energy delivered to a tissue treatment site is approximated to be 150 Joules for a 5 mm treatment length, 30 Joules of energy per mm per second would need to be delivered.

In a further example, a typical energy range may be between approximately 50 and 120 Joules. Accordingly, for every 5 mm of airway tissue, approximately 50 to 120 Joules of energy may be delivered. Therefore, approximately 10 to 24 Joules/mm of airway may be delivered. When energy delivery device 18 is moved at a faster rate, higher power levels may be required in order to achieve the desired energy dose for treatment. Conversely, when energy delivery device 18 is moved at a slower rate, lower power levels may be required in order to achieve the desired energy dose for treatment. Accordingly, the continuous real-time tracking of the movement and/or location of energy delivery device 18, in addition to any sensed or measured characteristics of the airway/tissue, provides feedback to energy generator 12, which may enable energy generator 12 to automatically adjust at least the energy parameter so as to apply customized energy delivery to the plurality of tissue treatment sites.

In an alternative embodiment, movement of energy delivery device 18 through a passageway may be controlled through an automated device, such as a robotic device (not shown) or any other suitable automatic device known to those skilled in the art. The robotic device may initially be set to move energy delivery device 18 at a constant speed through a passageway. The speed, however, may be automatically changed as energy delivery system 10 receives real-time feedback from energy delivery device 18 based on the sensed energy delivery parameters and the location of energy delivery device 18 during treatment.

Other embodiments of the present disclosure can be found in each of the references cited and incorporated by reference above and will be apparent to those skilled in the art from consideration of the specification and practice of the present disclosure disclosed herein. It is intended that the specification and examples be considered as exemplary only, with a true scope and spirit of the present disclosure being indicated by the following claims.

What is claimed is:

1. A method of treating tissue defining a passageway in a body, comprising:
positioning an energy delivery device in the passageway, wherein the energy delivery device includes an elongate member having an energy emitting portion at a distal portion of the elongate member;
contacting the tissue defining the passageway with the energy emitting portion;
moving the energy delivery device along the passageway;
real-time tracking a location and movement of the energy delivery device in the passageway; and
delivering energy to the tissue according to the real-time tracked location and movement of the energy delivery device as it is moved along the passageway.

2. The method of claim 1, wherein at least one energy delivery parameter is automatically adjusted as the energy delivery device is moved along the passageway.

3. The method of claim 1, wherein contact is maintained between the energy emitting portion and the tissue while the energy delivery device is moved along the passageway, and the real-time tracking includes generating an image of the passageway and imposing a real-time location of the energy delivery device on the image.

4. The method of claim 1, wherein the real-time tracking includes tracking the energy delivery device with a locating system and at least one locating implement.

5. The method of claim 4, wherein the at least one locating implement is attached to the energy delivery device.

6. The method of claim 2, wherein the at least one energy delivery parameter includes at least one of power, temperature, impedance and time.

7. The method of claim 2, wherein the energy emitting portion includes a plurality of legs forming a basket-type shape.

8. The method of claim 7, wherein at least one energy delivery parameter is varied along a length of at least one of the legs.

9. The method of claim 2, wherein the at least one energy delivery parameter is also adjusted in response to sensed characteristics of the tissue and/or passageway.

10. The method of claim 1, further comprising transitioning the energy emitting portion from a first, collapsed configuration to a second, expanded configuration and maintaining the energy emitting portion in the second, expanded configuration as the energy delivery device is moved along the passageway.

11. The method of claim 1, wherein the energy delivery device is moved along the passageway in a substantially continuous or discontinuous manner.

12. The method of claim 1, wherein the energy delivery device is moved along the passageway at a steady or unsteady rate.

13. An energy delivery system for treating tissue defining a passageway in a body, comprising:
an energy delivery device including an elongate member and an energy emitting portion at a distal portion of the elongate member;
at least one energy generator connected to the energy delivery device; and
a locating system configured to track the energy delivery device in real-time when the energy delivery device is within the passageway;
wherein the energy generator is configured to deliver energy via the energy emitting portion to the passageway according to the real-time tracking of the energy delivery device, and wherein the energy delivery system is configured to automatically adjust at least one energy delivery parameter as the energy delivery device is moved along the passageway.

14. The system of claim 13, wherein contact is maintained between the energy delivery device and tissue while the energy delivery device is moved along the passageway, and the locating system is configured to track the energy delivery device in real-time via communication with a locating implement located on the energy delivery device.

15. The system of claim 13, wherein the energy emitting portion includes a plurality of legs forming a basket-type shape.

16. The system of claim 15, wherein the energy delivery system is configured to vary the at least one energy delivery parameter along a length of at least one of the plurality of legs.

17. The system of claim 13, including a rendering system configured to generate an image of coordinates of the passageway.

18. The system of claim 13, wherein the energy delivery system is configured to also automatically adjust the at least one energy delivery parameter in response to measured characteristics of the tissue and/or passageway.

19. The system of claim 13, wherein the energy emitting portion is configured to transition from a first, collapsed configuration to a second, expanded configuration and wherein the energy emitting portion is configured to be maintained in the second, expanded configuration as the energy delivery device is moved along the passageway.

20. The system of claim 13, wherein the energy delivery system is configured to automatically adjust the at least one energy delivery parameter as a result of an inverse relationship between a rate of movement of the energy delivery device and an amount of energy delivered.
